(19) United States
(12) Patent Application Publication
(10) Pub. No.: US 2008/0102042 A1
(43) Pub. Date: May 1, 2008

(54) Oral Composition

(75) Inventors: Kenjiro Shimada, Ibaraki (JP); Toru Takahashi, Hokkaido (JP)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: KYOWA HAKKO KOGYO CO., LTD., TOKYO, JAPAN (JP)

(21) Appl. No.: 11/666,012

(22) PCT Filed: Oct. 28, 2005
(86) PCT No.: PCT/JP05/19872

§ 371 Date: Apr. 23, 2007

(30) Foreign Application Priority Data
Oct. 28, 2004 (JP) 2004-313262

(51) Int. Cl. A61K 31/401; A61K 8/49; A61Q 11/00; C07D 207/20

(52) U.S. Cl. 424/49; 514/423; 548/530

(57) ABSTRACT

The present invention provides an oral composition which is effective for the prevention or treatment of periodontal disease such as gingivitis, periodontitis and alveolar pyorrhea. According to the present invention, there can be provided an oral composition such as a dentifrice, a mouthwash or a gingival massage cream which is effective for the prevention or treatment of periodontal disease, said composition comprising an N-acyl derivative of hydroxyproline such as an N-acetyl derivative, an N-propionyl derivative, an N-butyryl derivative or an N-isobutyryl derivative, or a salt thereof.

### TECHNICAL FIELD

The present invention relates to an oral composition comprising an N-acyl derivative of hydroxyproline or a salt thereof.

### BACKGROUND ART

“Periodontal disease” is the general name for lesions developing at the periodontium and is usually broadly classified into gingivitis, in which the lesion is limited to the marginal gingiva, and periodontitis, which involves resorption of the alveolar bone. So-called alveolar pyorrhea is also called chronic progressive marginal periodontitis.

Known examples of therapeutic agents for periodontal disease include lysozyme chloride, vitamin E, vitamin C, glycyrrhizic acid, allantoin and hinokitiol (see patent document No. 1), but their effect is insufficient.

N-Acetylhydroxyproline is known to exhibit anti-inflammatory effect and particularly to be capable of acting on the metabolism of connective tissues of the joints, skin, cardiovascular system, etc. (see patent document No. 2). However, it is not known that N-acyl derivatives of hydroxyproline such as N-acetylhydroxyproline exhibit a preventive or therapeutic effect on periodontal disease.

### Patent document No. 1:

Japanese Published Unexamined Patent Application No. 12536/02

### Patent document No. 2:

U.S. Pat. No. 3,891,765

### DISCLOSURE OF THE INVENTION

### Problems to be Solved by the Invention

An object of the present invention is to provide an oral composition which is effective for the prevention or treatment of periodontal disease.

### Means for Solving the Problems

The present invention relates to the following (1) to (8).

(1) An oral composition for the prevention or treatment of periodontal disease comprising an N-acyl derivative of hydroxyproline or a salt thereof.

(2) The composition according to the above (1), wherein the N-acyl derivative of hydroxyproline is an N-acetyl derivative, an N-propionyl derivative, an N-butyryl derivative or an isobutyryl derivative.

(3) The composition according to the above (1) or (2), wherein the N-acyl derivative of hydroxyproline or a salt thereof is contained in an amount of 0.001 to 15% by weight.

(4) The composition according to any one of the above (1) to (3), wherein the oral composition is a dentifrice, a mouthwash or a gingival massage cream.

(5) A method of preventing or treating periodontal disease which uses an N-acyl derivative of hydroxyproline or a salt thereof.

(6) The method according to the above (5), wherein the N-acyl derivative of hydroxyproline is an N-acetyl derivative, an N-propionyl derivative, an N-butyryl derivative or an isobutyryl derivative.

(7) Use of an N-acyl derivative of hydroxyproline or a salt thereof for the manufacture of an oral composition for the prevention or treatment of periodontal disease.

(8) The use according to the above (7), wherein the N-acyl derivative of hydroxyproline is an N-acetyl derivative, an N-propionyl derivative, an N-butyryl derivative or an isobutyryl derivative.

### Effect of the Invention

The present invention provides an oral composition comprising an N-acyl derivative of hydroxyproline or a salt thereof which is effective for the prevention or treatment of periodontal disease.

### Best Modes for Carrying Out the Invention

The N-acyl derivatives of hydroxyproline used in the oral composition of the present invention can be prepared from hydroxyproline according to known methods.

Hydroxyproline may be any of its stereoisomers. That is, hydroxyproline can exist as eight kinds of stereoisomers according to whether proline is in the D or L form, whether the hydroxyl group is located at the 3- or 4-position, and whether the stereoisomer is in the cis or trans form, and any of these stereoisomers can be employed.

Specifically, hydroxyproline includes cis-4-hydroxy-L-proline, cis-4-hydroxy-D-proline, cis-3-hydroxy-L-proline, cis-3-hydroxy-D-proline, trans-4-hydroxy-L-proline, trans-4-hydroxy-D-proline, trans-3-hydroxy-L-proline and trans-3-hydroxy-D-proline.

Hydroxyproline is a kind of amino acid which exists widely in nature as a major amino acid component of collagen and as a constituent amino acid of elastin, and can be produced, for example, by acid hydrolysis of collagen derived from animals such as pig and cow, followed by purification by ordinary means.

Trans-4-hydroxy-L-proline can be produced by using proline 4-hydroxylase isolated from a microorganism of the genus Amycolatopsis or Dactylosporangium (Japanese Published Unexamined Patent Application No. 313179/95). Cis-3-hydroxy-L-proline can be produced by using proline 3-hydroxylase isolated from a microorganism of the genus Streptomyces (Japanese Published Unexamined Patent Application No. 322885/95) [Bioindustry, Vol. 14, No. 31 (1997)].

The above hydroxyprolines produced by using enzymes derived from microorganisms are superior in quality and are preferable as materials for preparing N-acyl derivatives.

N-Acyl derivatives of various kinds of stereoisomers of hydroxyproline mentioned above can be used as N-acyl derivatives of hydroxyproline for preparing the oral composition of the present invention.

The acyl group of the N-acyl derivatives includes acyl groups preferably having 1 to 24 carbon atoms, more preferably 1 to 12 carbon atoms, particularly preferably 1 to 6 carbon atoms. Examples of the acyl groups include formyl, acetyl, propionyl, butyryl, isobutyryl, valeryl, pivaloyl, hexanoyl, heptanoyl, octanoyl, nonanoyl, decanoyl, undecanoyl and dodecanoyl, and in particular, acetyl, propionyl, butyryl and isobutyryl are preferred.

The salts of the N-acyl derivatives of hydroxyproline include alkali metal salts such as sodium salt, potassium salt and lithium salt, alkaline earth metal salts such as calcium salt and magnesium salt, ammonium salt, amine addition salts such as salts with monoethanolamine, diethanolamine, triethanolamine and triisopropanolamine, and basic amino acid addition salts such as salts with arginine and lysine.

The N-acyl derivatives of hydroxyproline can be prepared by known methods. For example, the N-acyl derivatives of hydroxyproline can be produced by converting a saturated or unsaturated straight-chain or branched fatty acid having 1 to 24 carbon atoms into a halide (e.g., chloride or bromide) using a halogenating agent (e.g., thionyl chloride or phosgene) and then condensing the halide with the above-described hydroxyproline, or by converting the fatty acid into an acid anhydride and then reacting the acid anhydride with hydroxyproline.

Examples of the fatty acids include formic acid, acetic acid, propionic acid, butyric acid, isobutyric acid, valeric acid, isovaleric acid, pivalic acid, hexanoic acid, heptanoic acid, octanoic acid, nonanoic acid, decanoic acid, undecanoic acid and dodecanoic acid, which are used alone or in combination.

The method for producing an N-acyl derivative of hydroxyproline via an acid halide is described below.

A fatty acid is dispersed in a solvent such as methylene chloride, chloroform, carbon tetrachloride, benzene, toluene, xylene or n-hexane, and 1 to 5 equivalents of a halogenating agent is added thereto to carry out reaction, whereby a fatty acid halide is obtained. Subsequently, hydroxyproline is dissolved or dispersed in a solvent, and while keeping the resulting solution at 5 to 70° C., the above fatty acid halide is added in an amount of 0.3 to 3.0 equivalents based on hydroxyproline to carry out acylation reaction, whereby an N-acyl derivative of hydroxyproline can be produced.

Examples of the solvents used in the acylation reaction include water, methanol, ethanol, isopropanol, isobutanol, acetone, toluene, tetrahydrofuran, ethyl acetate, N,N-dimethylformamide and dimethyl sulfoxide, which may be used alone or as a mixture. When hydroxyproline is dissolved or dispersed in the solvent, an alkaline substance such as sodium hydroxide or potassium hydroxide (0.8 to 2.0 equivalents based on hydroxyproline) may be dissolved or dispersed in the solvent according to need.

When it is desired to obtain a salt of the N-acyl derivative of hydroxyproline, in the case where the N-acyl derivative of hydroxyproline is produced in the form of the salt, it can be subjected to purification as such, and where it is produced in the free form, it can be converted into a salt, after being dissolved or suspended in a suitable solvent, by adding a base thereto.

Purification is carried out, for example, by using ordinary methods such as crystallization and chromatography.

Specific examples of the N-acyl derivatives of hydroxyproline include N-acetyl-cis-4-hydroxy-L-proline, N-acetyl-cis-4-hydroxy-D-proline, N-acetyl-cis-3-hydroxy-L-proline, N-acetyl-cis-3-hydroxy-D-proline, N-acetyl-trans-4-hydroxy-L-proline, N-acetyl-trans-4-hydroxy-D-proline, N-acetyl-trans-3-hydroxy-L-proline, N-acetyl-trans-3-hydroxy-D-proline, N-propionyl-cis-4-hydroxy-L-proline, N-propionyl-cis-4-hydroxy-D-proline, N-propionyl-cis-3-hydroxy-L-proline, N-propionyl-cis-3-hydroxy-D-proline, N-propionyl-trans-4-hydroxy-L-proline, N-propionyl-trans-4-hydroxy-D-proline, N-propionyl-trans-3-hydroxy-L-proline, N-propionyl-trans-3-hydroxy-D-proline, N-butyryl-cis-4-hydroxy-L-proline, N-butyryl-cis-4-hydroxy-D-proline, N-butyryl-cis-3-hydroxy-L-proline, N-butyryl-cis-3-hydroxy-D-proline, N-butyryl-trans-4-hydroxy-L-proline, N-butyryl-trans-4-hydroxy-D-proline, N-butyryl-trans-3-hydroxy-L-proline, N-butyryl-trans-3-hydroxy-D-proline, N-isobutyryl-cis-4-hydroxy-L-proline, N-isobutyryl-cis-4-hydroxy-D-proline, N-isobutyryl-cis-3-hydroxy-L-proline, N-isobutyryl-cis-3-hydroxy-D-proline, N-isobutyryl-trans-4-hydroxy-L-proline, N-isobutyryl-trans-4-hydroxy-D-proline, N-isobutyryl-trans-3-hydroxy-L-proline, and N-isobutyryl-trans-3-hydroxy-D-proline.

In the oral composition of the present invention, the N-acyl derivatives of cis-4-hydroxy-L-proline, cis-4-hydroxy-D-proline, cis-3-hydroxy-L-proline, cis-3-hydroxy-D-proline, trans-4-hydroxy-L-proline, trans-4-hydroxy-D-proline, trans-3-hydroxy-L-proline or trans-3-hydroxy-D-proline or salts thereof can be used alone or as a mixture as the N-acyl derivative of hydroxyproline or a salt thereof.

The content of the N-acyl derivative of hydroxyproline or a salt thereof in the oral composition of the present invention is preferably 0.001 to 15% by weight, more preferably 0.01 to 15% by weight, and particularly preferably 0.1 to 15% by weight.

The oral composition of the present invention may take the form of a dentifrice (e.g., toothpaste and liquid dentifrice), a mouthwash, a gingival massage cream, a liquid or paste topical liniment, chewing gum, or the like. Preferred are a dentifrice, a mouthwash and a gingival massage cream.

The oral composition of the present invention may be formulated to comprise, in addition to the N-acyl derivative of hydroxyproline or a salt thereof, usual amounts of appropriate ingredients according to the kind of the composition and the like.

Examples of such ingredients include abrasives, binders, thickeners, surfactants, sweeteners, preservatives, flavors, coloring agents, humectants, solvents, and various kinds of active ingredients other than the N-acyl derivatives of hydroxyproline or salts thereof.

Examples of the abrasives include silica abrasives such as precipitated silica, silica gel, aluminosilicate and zirconosilicate, dipotassium hydrogenphosphate dihydrate, dipotassium hydrogenphosphate anhydride, calcium pyrophosphate, calcium carbonate, aluminum hydroxide, alumina, magnesium carbonate, magnesium tertiary phosphate, zeolite, zirconium silicate and synthetic resin abrasives.

Examples of the binders include cellulose derivatives such as sodium carboxycellulose and methyl cellulose, gums such as xanthane gum, tragacanth gum, karaya gum and gum arabic, and synthetic binders such as polyvinylpyrrolidone.

Examples of the thickeners include glycerin, sorbitol, propylene glycol, polyethylene glycol, xylitol, maltitol and lactitol.

The surfactants include anionic surfactants, cationic surfactants, nonionic surfactants, etc., specifically, sodium lauryl sulfate, sodium α-olefin sulfonate, N-acyl salcosinate, N-acyl glutamate, 2-alkyl-N-carboxymethyl-N-hydroxyethyl imidazolinium betaine, N-acyl taurate, sucrose fatty acid ester, alkylol amide, polyoxyethylene hardened castor oil, aliphatic ester of polyglycerin, Pluronic, polyoxyethylene sorbitan monostearate, etc.

Examples of the sweeteners include sodium saccharine, stevioside, stevia extract, paramethoxycinnamic aldehyde, neohesperidyl dihydrochalcone and perillartine.

Examples of the preservatives include paraoxybenzoic acid ester and sodium benzoate.

Examples of the flavors include terpenes such as 1-menthol, carvone, anethol and limonene, and their derivatives.

Examples of the coloring agents include Brilliant Blue FCF, Tartrazine and titanium dioxide.

Examples of the humectants include glycerin, sorbitol and polyethylene glycol.

Examples of the solvents include ethanol and hexylene glycol.

Examples of the various active ingredients include fluorides such as sodium fluoride, potassium fluoride, ammonium fluoride, stannous fluoride and sodium monofluorophosphate, water-soluble phosphoric acid compounds such as potassium salt and sodium salt of orthophosphoric acid, allantoin chlorohydroxy aluminum, hinokitiol, ascorbic acid, lysozyme chloride, glycyrrhizinic acid and salts thereof, sodium chloride, tranexamic acid, epsilon-aminocaproic acid, dl-tocopherol acetate, azulene, glycyrrhetic acid, copper compounds such as sodium copper chlorophyllin and copper gluconate, aluminum lactate, strontium chloride, potassium nitrate, berberine, hydroxamic acid and derivatives thereof, sodium tripolyphosphate, zeolite, dextranase, mutanase, amylase, methoxyethylene-maleic anhydride copolymer, polyvinylpyrrolidone, epidihydrocholesterin, dihydrocholesterol, zinc citrate, extracts of ligusticum, Phellodendron bark, clove, rosemary, scutellaria, safflower, etc., α-bisabolol, chlorohexidine salts, cetyl pyridinium chloride, benzetonium chloride, and trichlorocarbanilide.

By daily use of the oral composition of the present invention, periodontal disease can be prevented.

“To prevent periodontal disease” means to achieve effects such as complete prevention of incidence of periodontal disease, reduction of incidence of periodontal disease and suppression of symptoms of periodontal disease at the incidence thereof by daily use of the oral composition of the present invention.

In the case where periodontal disease already manifested itself, the disease can be treated by daily use of the oral composition of the present invention.

“To treat periodontal disease” means to achieve effects such as improvement or cure of the symptoms accompanying periodontal disease by daily use of the oral composition of the present invention after the disease advanced.

The amount of the oral composition of the present invention to be used and the frequency of use thereof vary depending on the kind of the composition, the symptoms, etc., but it is preferable to use the composition in an amount of 0.01 mg to 1 g, more preferably 0.1 mg to 1 g, particularly preferably 1 mg to 1 g in terms of the N-acyl derivative of hydroxyproline or a salt thereof per use once to five times per day.

“Periodontal disease” is the general name for lesions developing at the periodontium and includes gingivitis, in which the lesion is limited to the marginal gingiva, and periodontitis, which involves resorption of the alveolar bone. So-called alveolar pyorrhea is another name for chronic progressive marginal periodontitis, which is one of the pathological states of periodontal disease.

The oral composition of the present invention can be used for the prevention or treatment of periodontal disease not only in humans but also in non-human animals such as dogs and cats.

Shown below are test examples on the preventive or therapeutic effect of N-acetyl-trans-4-hydroxy-L-proline (hereinafter referred to also as N-acetylhydroxyproline) on periodontal disease.

### TEST EXAMPLE 1

Buffered saline (pH 7.0) containing 2.5% by weight of N-acetylhydroxyproline (0.8 ml) was injected into the periodontal pocket of a patient with advanced alveolar pyorrhea and the gingiva was lightly massaged. This treatment was carried out twice a day for two weeks, and as a result, the symptoms of alveolar pyorrhea were remarkably improved.

The above result shows the therapeutic effect of N-acetylhydroxyproline on periodontal disease.

### TEST EXAMPLE 2

Buffered saline (pH 7.0) containing 2.5% by weight of N-acetylhydroxyproline (0.8 ml) was injected into the periodontal pocket of a patient with alveolar pyorrhea who had a big periodontal pocket and a loose tooth, and the gingiva was lightly massaged.

This treatment was carried out after toothbrushing three times a day for two weeks. As a result, the pain of alveolar pyorrhea completely disappeared and the looseness of the tooth was remarkably improved.

Thereafter, toothbrusing was continuously carried out using a very fine toothbrush with buffered saline (pH 7.0) containing 2.5% by weight of N-acetylhydroxyproline on it with massage of the gingiva and the inside of the periodontal pocket. This toothbrusing was carried out for more than three minutes three times a day.

As a result, the healthy state without pain or looseness of tooth could be maintained.

The above result shows not only the therapeutic effect of N-acetylhydroxyproline on periodontal disease but also its effect of preventing the recurrence of periodontal disease, i.e., the preventive effect on periodontal disease.

Certain embodiments of the present invention are illustrated in the following examples.

### EXAMPLE 1

### Dentifrice (1)

A dentifrice comprising N-acetyl-trans-4-hydroxy-L-proline which has the following composition is produced according to a conventional method.

| Ingredient | Amount (wt %) |
|---|---|
| N-Acetyl-trans-4-hydroxy-L-proline | 5.0 |
| Calcium secondary phosphate dihydrate | 40.0 |
| Silicic acid anhydride | 5.0 |
| Glycerin | 10.0 |
| Sorbitol | 5.0 |
| Sodium carboxymethylcellulose | 1.0 |
| Carrageenan | 0.3 |
| Sodium lauryl sulfate | 1.4 |
| Arginine | 2.5 |
| Dipotassium glycyrrhizinate | 0.1 |
| Sodium saccharine | 0.1 |
| Ethyl paraoxybenzoate | 0.05 |
| Flavor | 0.5 |
| Purified water | 29.05 |

### EXAMPLE 2

### Dentifrice (2)

A dentifrice comprising N-propionyl-trans-4-hydroxy-L-proline which has the following composition is produced according to a conventional method.

| Ingredient | Amount (wt %) |
|---|---|
| N-Propionyl-trans-4-hydroxy-L-proline | 5.0 |
| Silicic acid anhydride | 20.0 |
| Sorbitol solution | 30.0 |
| Glycerin | 10.0 |
| Sodium carboxymethylcellulose | 1.5 |
| Sodium lauryl sulfate | 1.0 |
| 1-Menthol | 0.1 |
| Ethyl paraoxybenzoate | 0.1 |
| Sodium saccharine | 0.1 |
| Flavor | 0.5 |
| Purified water | 31.7 |

### EXAMPLE 3

### Dentifrice (3)

A dentifrice comprising N-butyryl-trans-4-hydroxy-L-proline which has the following composition is produced according to a conventional method.

| Ingredient | Amount (wt %) |
|---|---|
| N-Butyryl-trans-4-hydroxy-L-proline | 5.0 |
| Propylene glycol | 3.0 |
| 70% Sorbitol | 20.0 |
| Sodium carboxymethylcellulose | 0.8 |
| Silica | 20.0 |
| Saccharine | 0.3 |
| Sodium lauryl sulfate | 1.0 |
| Flavor | 1.0 |
| Purified water | 48.9 |

### EXAMPLE 4

### Mouthwash (1)

A mouthwash comprising N-acetyl-trans-4-hydroxy-L-proline which has the following composition is produced according to a conventional method.

| Ingredient | Amount (wt %) |
|---|---|
| N-Acetyl-trans-4-hydroxy-L-proline | 2.5 |
| Ethanol | 10.0 |
| Glycerin | 20.0 |
| Polyoxyethylene hardened castor oil | 0.05 |
| Flavor | 0.8 |
| Sodium fluoride | 0.2 |
| Purified water | 66.45 |

### EXAMPLE 5

### Mouthwash (2)

A mouthwash comprising N-propionyl-trans-4-hydroxy-L-proline which has the following composition is produced according to a conventional method.

| Ingredient | Amount (wt %) |
|---|---|
| N-Propionyl-trans-4-hydroxy-L-proline | 2.5 |
| Ethanol | 25.0 |
| Glycerin | 10.0 |
| Sodium benzoate | 0.3 |
| Polyoxyethylene hardened castor oil | 2.0 |
| Citric acid | 0.5 |
| Sodium citrate | 0.5 |
| Sodium saccharine | 0.1 |
| Flavor | 0.3 |
| Purified water | 58.8 |

### EXAMPLE 6

### Gingival Massage Cream

A gingival massage cream comprising N-acetyl-trans-4-hydroxy-L-proline which has the following composition is produced according to a conventional method.

| Ingredient | Amount (wt %) |
|---|---|
| N-Acetyl-trans-4-hydroxy-L-proline | 2.5 |
| White vaseline | 8.0 |
| Propylene glycol | 4.0 |
| Carrageenan | 0.5 |
| Sodium carboxymethylcellulose | 0.7 |
| Polyethylene glycol 400 | 35.0 |
| Polyoxyethylene hardened castor oil | 2.0 |
| Flavor | 0.3 |
| Purified water | 47.0 |

### INDUSTRIAL APPLICABILITY

The present invention provides an oral composition comprising an N-acyl derivative of hydroxyproline or a salt thereof which is effective for the prevention or treatment of periodontal disease.

1. An oral composition for the prevention or treatment of periodontal disease comprising an N-acyl derivative of hydroxyproline or a salt thereof.

2. The composition according to claim 1, wherein the N-acyl derivative of hydroxyproline is an N-acetyl derivative, an N-propionyl derivative, an N-butyryl derivative or an N-isobutyryl derivative.

3. The composition according to claim 1 or 2, wherein the N-acyl derivative of hydroxyproline or a salt thereof is contained in an amount of 0.001 to 15% by weight.

4. The composition according to any one of claims 1 to 3, wherein the oral composition is a dentifrice, a mouthwash or a gingival massage cream.

5. A method of preventing or treating periodontal disease which uses an N-acyl derivative of hydroxyproline or a salt thereof.

6. The method according to claim 5, wherein the N-acyl derivative of hydroxyproline is an N-acetyl derivative, an N-propionyl derivative, an N-butyryl derivative or an N-isobutyryl derivative.

7. Use of an N-acyl derivative of hydroxyproline or a salt thereof for the manufacture of an oral composition for the prevention or treatment of periodontal disease.

8. The use according to claim 7, wherein the N-acyl derivative of hydroxyproline is an N-acetyl derivative, an N-propionyl derivative, an N-butyryl derivative or an N-isobutyryl derivative.
